(19) United States
(12) Patent Application Publication
(10) Pub. No.: US 2018/0104456 A1
(43) Pub. Date: Apr. 19, 2018

(54) VENOUS ANATOMY DRUG DELIVERY BALLOON

(71) Applicant: Divergent Life Sciences LLC, Germantown, TN (US)

(72) Inventor: Gary R. DAVIS, Germantown, TN (US)

(73) Assignee: Divergent Life Sciences LLC, Germantown, TN (US)

(21) Appl. No.: 15/786,287

(22) Filed: Oct. 17, 2017

Related U.S. Application Data
(60) Provisional application No. 62/409,009, filed on Oct. 17, 2016.

(51) Int. Cl. A61M 25/10; A61L 29/16

(52) U.S. Cl. CPC A61M 25/104; A61L 29/16; A61F 2250/0067; A61M 25/1018; A61M 2025/1052; A61M 25/1011

(57) ABSTRACT

Balloon catheters that deliver therapeutic agents are functional only for treatment of the arterial anatomy and result in inefficient dispersion of the therapeutic agent to the arterial tissue due to dispersion through the secondary balloon holes under high pressure. A method and drug delivery balloon device for efficient drug delivery to the targeted point of the anatomy comprising a first balloon wholly enveloped by a second balloon composed of a porous membrane that delivers the therapeutic agent to the targeted point upon the introduction of low pressure to the secondary balloon membrane. In the venous anatomy, the drug delivery balloon device interacts directly with the diseased, narrow segment of the venous vessel while not damaging the vessel tissue. Further, in treatment of arteriovenous (AV) fistulas, the drug delivery balloon device allows the physician to choose the appropriate drug and quantity for treating the narrowing (smooth muscle hyperplasia) or blood clots (thrombus burden) in the fistula.

### CROSS-REFERENCES TO RELATED APPLICATIONS

This application claims the benefit of U.S. Provisional Application No. 62/409,009, filed Oct. 17, 2016, which is incorporated herein by reference in its entirety.

### FIELD OF THE INVENTION

The present invention is generally directed toward a drug delivery balloon device. More particularly, the present invention relates to the delivery of a therapeutic agent via a drug delivery balloon device to treat the venous anatomy and arteriovenous fistulas.

### BACKGROUND OF THE INVENTION

Drug delivery balloon devices, such as balloon catheters, that administer therapeutic agents within the arterial anatomy are known in the art. These current devices deliver the necessary therapeutic agents via high pressure diffusion through holes in the outer balloon of the drug delivery balloon device. More specifically, the balloon catheters are configured to include an inflatable first balloon at least partially enclosed by an expandable second balloon that has holes for delivery of the therapeutic agent to the targeted site of the arterial anatomy. The annular space between the balloons is in communication with the holes of the second balloon and configured to receive and then release and distribute the therapeutic agent via the secondary balloon holes. The release of the therapeutic agent via these holes can result in release of more of the therapeutic agent than is necessary or uneven delivery into a particular portion of the arterial tissue resulting in extensive tissue damage. Therefore, a more efficient, direct delivery method is needed.

Generally, the devices known in the art are used to perform percutaneous transluminal angioplasty (PTA) procedures where the drug delivery balloon device is threaded into and through an arterial vessel lumen along a wire guide and positioned at a stenosis or other point of treatment, where the balloon is inflated to dilate the target site of the arterial vessel lumen. After treatment, the balloon is deflated and the catheter is removed from the target site and the patient's lumen. While the current devices treat the arterial anatomy, a need exists for a drug delivery balloon device that treats the venous anatomy.

Also, there are approximately 680,000 patients in the U.S. with surgically created arteriovenous (AV) fistulas receiving hemodialysis. Over 60% of these fistulas (access sites) require additional treatment and intervention during the life of the patient, for narrowing and blood clots. This is estimated to add an additional $2.8 billion to the cost of treating these patients and is driven by the necessity for re-intervention. Currently there is no technology that gives physicians a good tool for lowering these interventions rates. Typically patients are treated as many as three times a year for AV fistula narrowing, with some patients requiring as many eight treatments per year to maintain AV fistula function. Reducing re-intervention rates will improve the quality of life for the patient and lower cost significantly. Recently, the FDA approved the use of drug coated balloons (DCBs) for the treatment of AV fistulas. DCBs are expensive ($1,295 to $1,995) and do not offer any quantitative data on the amount of drug delivered, nor do they offer the physician a choice in the type of drug delivered. Thus a need exists for a device which allows the physician to choose the appropriate drug and quantity for treating the narrowing (smooth muscle hyperplasia) or blood clots (thrombus burden) in the fistula.

### SUMMARY OF THE INVENTION

Drug delivery balloons that deliver therapeutic agents are functional only for the arterial anatomy and result in inefficient dispersion of the therapeutic agent to arterial tissue due to dispersion through the secondary balloon holes. The presently disclosed invention is a drug delivery balloon device comprising a balloon assembly mounted on a standard catheter shaft, wherein the balloon assembly further comprises a first balloon wholly enveloped by a second balloon that delivers the therapeutic agent to the venous anatomy upon the introduction of pressure to the porous membrane of the second balloon, thus interacting directly with the diseased and/or narrow segment of the vessel. The drug delivery balloon device is also functional for the treatment of arteriovenous (AV) fistulas. The porous membrane of the second or outer balloon of the balloon assembly allows for efficient, even distribution of the therapeutic agent to the targeted point of the anatomy under low pressure, resulting in increased patient outcomes.

### BRIEF DESCRIPTION OF THE DRAWINGS

Further advantages of the invention will become apparent by reference to the detailed description of preferred embodiments when considered in conjunction with the drawings:

FIG. 1 depicts a perspective view of the drug delivery balloon device.

FIG. 2 depicts a longitudinal cross sectional view of the drug delivery balloon device.

FIG. 3 depicts a transverse cross sectional view of the drug delivery balloon device.

FIG. 4 depicts a longitudinal cross sectional view of the drug delivery balloon device.

FIG. 5 depicts a perspective view of the catheter hub of the drug delivery balloon device.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown, but is to be accorded the widest possible scope consistent with the principles and features disclosed herein.

Drug delivery balloon devices known in the art disperse therapeutic agent to arterial vessels under high pressure via holes in the secondary balloon of the balloon assembly, which can result in release of more of the therapeutic agent than is necessary or uneven delivery into a particular portion of the arterial tissue resulting in extensive tissue damage. Further, these devices are limited to treatment of arterial vessels due to the significantly smaller size arteries than veins. Thus a more efficient, direct delivery device and method is needed which treats both arteries and veins.

Generally, the devices known in the art are used to perform percutaneous transluminal angioplasty (PTA) procedures where the drug delivery balloon device is threaded into and through an arterial vessel lumen along a wire guide and positioned at a stenosis or other point of treatment, where the balloon is inflated to dilate the target site of the arterial vessel lumen. After treatment, the balloon is deflated and the catheter is removed from the target site and the patient's lumen. While the current devices treat the arterial anatomy, a need exists for a drug delivery balloon device that treats the venous anatomy.

The present invention is a drug delivery balloon device 10 and method for delivering a therapeutic agent to a specific diseased portion of the venous anatomy upon the introduction of pressure. The drug delivery balloon device 10 solves the problem of inefficient dispersion of the therapeutic agent to body tissue due to dispersion through the secondary balloon holes by using a porous membrane upon the induction of lower pressure. In the venous anatomy, the drug delivery balloon device interacts directly with the diseased, narrow segment of the venous vessel while not damaging the vessel tissue. The increased precision of the present device results in better patient outcomes. Further, in contrast to the prior art which is only able to deliver a therapeutic agent to the arterial anatomy, the present device is configured to treat larger caliber venous vessels averaging from 12 to 20 millimeters in diameter compared to arterial vessels averaging 2-7 millimeters in diameter, as well as, arteriovenous (AV) fistulas.

The drug delivery balloon device 10 consists of a balloon assembly 11 mounted on a catheter shaft 12. The balloon assembly 11 further comprises a first, primary balloon 20 mounted on the catheter shaft 12 and connected to a guide wire lumen 22 wholly enveloped by a second balloon 21 comprised of a porous material. The second balloon 21 is filled with a therapeutic agent, which is delivered under pressure to the second balloon 21 and then permeates across the porous secondary balloon membrane 25 and subsequently interacts directly with the diseased or narrowed segment of the vessel. The “therapeutic agent” as used herein refers to any liquid capable of being transferred to a patient's vessel via the drug delivery balloon device 10.

In the preferred embodiment of the invention, the contemplated catheter shaft 12 comprises three tubular shaped lumens: a conventional guide wire lumen 22, an inflation lumen 23, and a fluid delivery lumen 24. As shown in FIG. 1, the drug delivery balloon device 10 extends from a proximal end 14 to a distal end 16. Therebetween, the drug delivery balloon device 10 includes a proximal region 13 of the catheter shaft 12 which includes a manifold 17 and a distal region 15 which includes the balloon assembly 11. The manifold 17 is joined to a traditional catheter shaft 12 and includes the corresponding commercially available guide wire port 28, injection port 26, and inflation port 29, as shown in FIG. 5.

The balloon assembly 11 is operated via a traditional “over the wire” system with the guide wire port 19 positioned as part of the manifold 17, which provides access to the guide wire lumen 22 extending through the catheter shaft 12 from the guide wire port 28. This guide wire lumen 22 of the catheter shaft 12 is configured to house a guide wire 18, including but not limited to, both 0.014 mm and 0.018 mm guide wires.

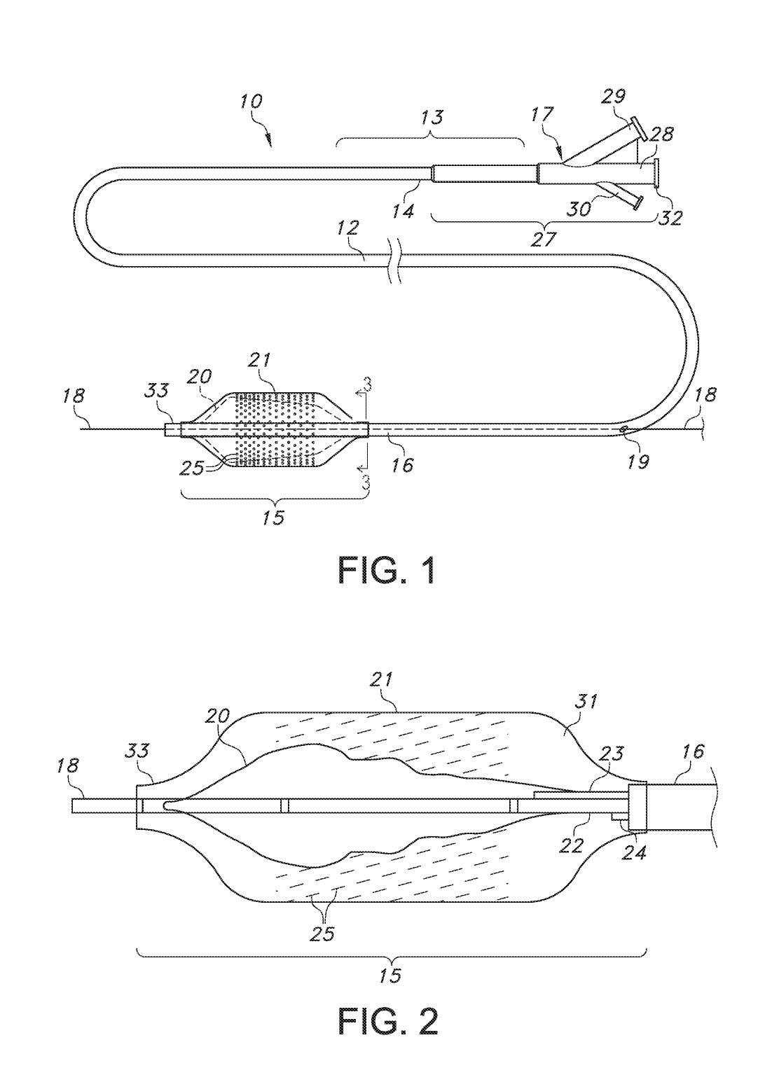

The distal region 15 of the drug delivery balloon device 10, as depicted in FIG. 2 includes the balloon assembly 11 comprised of a first balloon 20 and second balloon 21 carried on the catheter shaft 12. The first balloon 20 includes the guide wire lumen extended from the catheter shaft 12 and an inflation lumen 23, which communicates with the balloon 20 for purposes of inflation and deflation. The second balloon 21 is ultrasonically welded to the first balloon 20 and composed of a poreflon porous membrane 25 which is positioned to wholly envelop the first balloon 20 at the distal end 16 of the catheter shaft 12. The second balloon 21 is in fluid communication with the injection port 26 through the catheter shaft 12 and separate from the first balloon 20 and inflation port 29.

Individually, this first balloon 20 includes both an inflation lumen 23 and fluid delivery lumen 24 as shown in FIG. 4. The inflation lumen 23 communicates with the first balloon 20 and second balloon 21 for the purpose of inflation and deflation and is configured to house a traditional guide wire 18. The fluid delivery lumen 24 is for the delivery of a therapeutic agent to the annular space 27 created between the first balloon 20 and second balloon 21. The present invention contemplates a combined diameter of the balloon assembly 11 from 1.50 to 10.0 millimeters with lengths ranging from 20 to 200 millimeters.

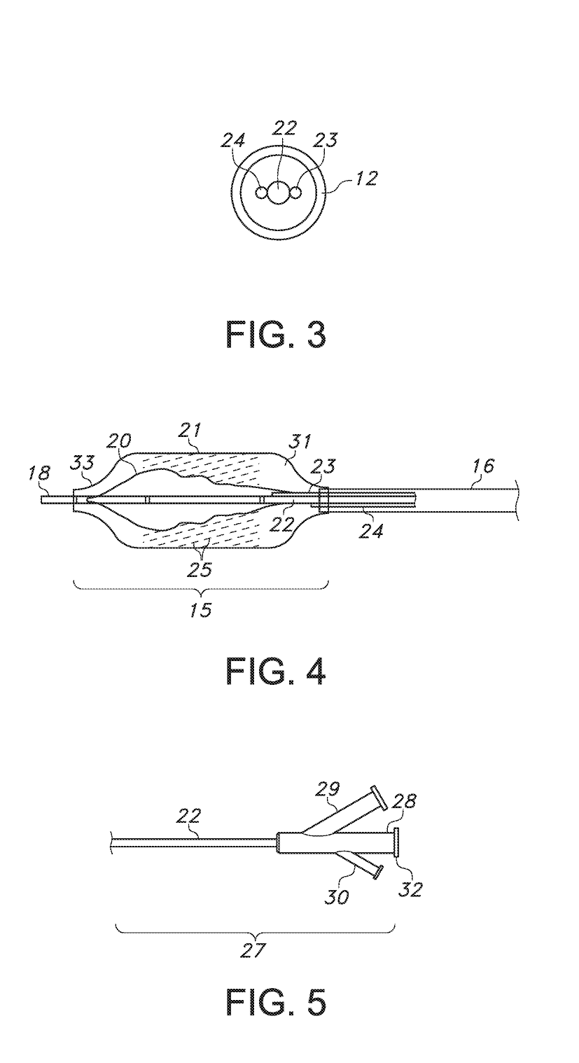

As shown in FIG. 3, the preferred embodiment of the catheter shaft 12 carries the three lumens which extend through the catheter shaft 12 to the balloon assembly 11. The guide wire lumen 22 measures 0.018 millimeters in diameter, while the inflation lumen 23 and fluid delivery lumen 24 measure 0.016 millimeters in diameter. However, lumens of any size capable of effectively communicating with the balloon assembly 11 and delivering the therapeutic agent to the vasculature are contemplated by this drug delivery balloon device 10.

The porous membrane 25 of the second balloon 21 delivers fluid to the venous vessel wall and is designed not to cause tissue damage. It is slightly longer than the first balloon 20 and carried on the same catheter shaft 12. Instead of dispersing the therapeutic agent through holes in the second balloon under high pressure like balloon catheters of the prior art, the proposed drug delivery balloon device 10 delivers the therapeutic agent to the venous vasculature via pressure introduced into the annular space 27 between the first balloon 20 and second balloon 21.

The first balloon 20 can be comprised of any standard material, such as polytetrafluoroethylene (PTFE), PEBAX, or nylon. The second balloon 21, on the other hand, is composed of any porous material capable of delivering the therapeutic agent to the venous vessel wall under low pressure, such as poreflon.

In the preferred embodiment, the catheter shaft 12 is threaded into and through the venous vessel lumen along the conventional guide wire 18 and positioned at a narrowing or diseased point of treatment in a patient's venous vessel, where the balloon assembly 11 is inflated to an appropriate pressure to dilate the target point of the body vessel lumen, approximately 80-90% of the vessel diameter. The therapeutic agent is then introduced to the annular space 27 located between the first balloon 20 and outer second balloon 21 via the fluid delivery lumen 24 under 1 to 2 atmospheres of pressure.

Specifically, upon uniform dispersion of the fluid, the inflation lumen 23 is inflated to the previous pressure. This fluid dispersion and inflation action causes the therapeutic agent to permeate across the porous membrane 25 of the second balloon 21 into the wall of the venous vessel in a safe and quantitative manner via the porous membrane 25. The liquid migrates across the porous membrane 25 at the pressure of one to two atmospheres, thus eliminating tissue damage witnessed in the prior art. The porous membrane inflates to a diameter of 10% of the first balloon 20 when fluid is introduced at 1 to 2 atmospheres of pressure. This introduction of pressure causes the fluid to permeate across the porous membrane 25 into the venous vessel wall. This process can be repeated as many times as necessary to treat the narrowing or diseased portion of the vessel. After treatment, the balloon assembly 11 is deflated and the drug delivery balloon device 10 is removed from the target point and the patient's lumen.

The preferred embodiment of the drug delivery balloon device 10 is proposed for use in the venous anatomy. However, the drug delivery balloon device 10 can also be utilized to treat segments in the arterial anatomy and provides improvements to the prior art with the utilization of lower pressure and effective distribution of the therapeutic agent to the vessel wall across the porous membrane 25 of the second balloon 21.

While the preferred embodiment of the invention delivers the therapeutic agent to the vessel wall at a pressure of one to two atmospheres, any pressure low enough to dilate the target point of the body vessel lumen and not damage the issue is contemplated by this drug delivery balloon device 10.

As discussed above, re-interventions are a common problem and hinder treatment in patients on hemodialysis. While drug coated balloons are approved by the FDA for treatment of arteriovenous (AV) fistulas, this form of treatment is extremely costly and limits the physicians choice of drug and quantity of said drug. In another embodiment, the drug delivery balloon device 10 is contemplated for the treatment of arteriovenous fistulas to address the need for a more cost effective method. The cost of treating arteriovenous fistulas using the drug delivery balloon device 10 would be approximately 1/10th of the cost associated with using drug coated balloons. Moreover, use of the drug delivery balloon device 10 allows the physician to choose the appropriate drug for treating the narrowing (smooth muscle hyperplasia) or blood clots (thrombus burden) in the fistula.

Specifically, a balloon assembly 11 ranging in diameter from 6 to millimeters and length from 30 to 50 millimeters is mounted on a catheter shaft 12 45 centimeters long with a 0.035 millimeter guide wire 18 to deliver the therapeutic agent to the target point of the arteriovenous fistula. However, as should be appreciated, any sized balloon assembly 11, catheter shaft 12, and guide wire 18 that allow for delivery of therapeutic agent to the targeted site of the narrowing or blood clots of the fistula is contemplated herein. Further, this embodiment is not limited to treatment of arteriovenous fistulas surgically created for use in dialysis, and is functional for treatment of arteriovenous fistulas anywhere in the body occuring by any means.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all subranges and individual values are intended to be encompassed. This invention is not to be limited by the embodiments disclosed, including any shown in the drawings or exemplified in the specification, which are given by way of example and not of limitation.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

I claim:

1. A drug delivery balloon device for evenly dispersing therapeutic agents to a patient's vessel comprising:
a. a catheter shaft;
b. a balloon assembly located at the proximal end of the catheter shaft further comprising a first balloon and second balloon, wherein said first balloon is wholly enveloped by a second balloon and carried on said catheter shaft;
c. a manifold located at the distal end of the catheter shaft further comprising a guide wire port, injection port, and inflation port; and a
d. guide wire.

2. The second balloon of claim 1 wherein said second balloon has a porous membrane.

3. The porous membrane of claim 2 wherein said porous membrane is poreflon.

4. The drug delivery balloon device of claim 1 wherein said drug delivery balloon device delivers therapeutic agent to a vessel wall of the venous anatomy.

5. The drug delivery balloon device of claim 1 wherein said drug delivery balloon device delivers therapeutic agent to the arterial anatomy.

6. The drug delivery balloon device of claim 1 wherein said drug delivery balloon device treats arteriovenous (AV) fistulas.

7. The drug delivery balloon device of claim 1 wherein said drug delivery balloon device delivers the therapeutic agent to a vessel wall at a pressure of 1 to 2 atmospheres.

8. A method for delivering therapeutic agent to a patient comprising:
a. threading a drug delivery balloon device comprising a first and second balloon through a patient's vessel lumen along a guide wire, wherein said second balloon wholly envelops the first balloon and is mounted on a catheter shaft;
b. positioning the catheter shaft at a target point of treatment in a patient's vessel;
c. inflating the balloon assembly to an appropriate pressure to dilate the target point of the vessel; and
d. introducing the therapeutic agent to target point of treatment under pressure via the porous membrane of the second balloon.

9. The porous membrane of claim 8 wherein said porous membrane of the second membrane is poreflon.

10. The drug delivery balloon device of claim 8 wherein said drug delivery balloon device delivers therapeutic agent to the venous anatomy.

11. The drug delivery balloon device of claim 8 wherein said drug delivery balloon device delivers therapeutic agent to the arterial anatomy.

12. The drug delivery balloon device of claim 8 wherein said drug delivery balloon device treats arteriovenous (AV) fistulas.

13. The pressure of claim 8 wherein said pressure dilates the target site of the body vessel lumen to 80-90% of the vessel diameter.

14. The vessel of claim 8 wherein said vessel diameter is 12 to 20 millimeters.

15. The pressure of claim 8 wherein said pressure is 1 to 2 atmospheres.
